(19) United States
(12) Patent Application Publication
(10) Pub. No.: US 2022/0061636 A1
(43) Pub. Date: Mar. 3, 2022

(54) STABILIZATION AND LEVERAGE DEVICES, SYSTEMS, AND METHODS

(71) Applicant: Boston Scientific Scimed, Inc., Maple Grove, MN (US)

(72) Inventors: Kathryn Venuto, Worcester, MA (US); Paul Smith, Smithfield, RI (US)

(21) Appl. No.: 17/405,764

(22) Filed: Aug. 18, 2021

Related U.S. Application Data
(60) Provisional application No. 63/071,412, filed on Aug. 28, 2020.

(51) Int. Cl. A61B 1/00; A61B 1/018

(52) U.S. Cl. CPC A61B 1/00098; A61B 17/29; A61B 1/00082; A61B 1/018

(57) ABSTRACT

According to embodiments of the present disclosure, a stabilizer is configured to engage and stabilize a target tissue site so that a procedure may be performed on a target tissue independently of the stabilizer. A lever is associated with the stabilizer and coupled with the target tissue to leverage the target tissue. In some embodiments, the stabilizer is deployed proximal to the target tissue site, with the lever proximal to the target tissue site and proximal to the distal end of the delivery device used to deploy the stabilization and leverage device. In such embodiment, the lever includes a lever component which may extend through a passage in the stabilizer to be controlled at a proximal end of the delivery device.

### PRIORITY

The present application is a non-provisional of, and claims the benefit of priority under 35 U.S.C. § 119 to, U.S. Provisional Application Ser. No. 63/071,412, filed Aug. 28, 2020, the disclosure of which is hereby incorporated herein by reference in its entirety for all purposes.

### FIELD

The present disclosure relates generally to the field of medical instruments and devices. More particularly, the present disclosure relates to medical instruments and devices for use in medical applications, such as, for example, manipulating body tissue during procedures on the tissue such as resection or dissection.

### BACKGROUND OF THE DISCLOSURE

A number of medical procedures utilize medical devices or tools within the body (within the digestive tract, the abdominal cavity, the biliary tract, the thoracic cavity, etc.) to access tissue intended for removal (e.g., “target tissue”). For example, in some current medical procedures (e.g., endoscopic submucosal dissection (ESD), endoscopic mucosal resection (EMR), Peroral Endoscopic Myotomy (POEM), cholecystectomy, Video-Assisted Thoracoscopic Surgery (VATS)), physician or clinicians (used interchangeably herein) may utilize an endoscope or similar medical device to access and remove diseased lesions.

With regard to endoscopic procedures, physicians have become increasingly willing to perform aggressive interventional and therapeutic procedures, such as for the removal of larger lesions (both cancerous and noncancerous). While physicians are becoming more proficient at resecting and dissecting diseased lesions from within the body (e.g., within the digestive tract, abdominal cavity, thoracic cavity, etc.), and specialized tools allow the physician to perform these complex procedures faster and easier, there are still a number of challenges. Such procedures may include multiple device exchanges. Endoscopes provide little working space and stabilization for multiple tools to operate simultaneously.

It is with these considerations in mind that the improvements in tissue stabilization and leverage devices, systems, and related methods of use presented by the present disclosure may be useful.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary. Accordingly, while the disclosure is presented in terms of aspects or embodiments, it should be appreciated that individual aspects can be claimed separately or in combination with aspects and features of that embodiment or any other embodiment.

The present disclosure, in its various aspects, is directed generally to medical devices, and more specifically to tissue stabilization and leverage devices, methods, and related systems. Embodiments according to the present disclosure, including as described herein, may decrease complications around procedures performed on body tissues, such as visualization, procedure time, and procedure complexity. Systems, devices, and methods disclosed herein may be compatible with other devices, systems, and methods, such as used with an endoscope. In some embodiments, such systems, devices, and methods may operate independently of the endoscope so that when the endoscope is being manipulated to perform a procedure on the tissue (e.g., to retract or to cut tissue), the stabilization and leverage mechanisms remain fixed and unaffected by the movement of the endoscope and tools used for the procedure. In some embodiments, the systems and devices disclosed herein may leverage and/or retract tissue independently of the movement of the delivery device and/or other tools to be used on the tissue.

In an aspect, a stabilization and leverage device is disclosed herein. The device may be configured for delivery, by a delivery device, to a target tissue at a target tissue site. In some aspects, the stabilization and leverage device includes a stabilizer configured for mounting on a delivery device, and a lever associated with the stabilizer and extending between the target tissue and the stabilizer to leverage the target tissue. The stabilizer contacts and stabilizes the target tissue independently of the distal end of the delivery device to allow independent free movement of the delivery device and instruments extended therethrough relative to the stabilizer and the target tissue. In some embodiments, the stabilizer may be configured for being loaded over the delivery device.

In some aspects, the lever comprises one or more of: a support elevator; a tensioner or controller; a lever tool; a direction-changing structure; a tissue traction device.

In some aspects, a stabilizer passage extends from a proximal end to a distal end of the stabilizer; and the lever includes a lever component extending from a first end coupled with the target tissue, through the stabilizer passage, to a second end controllable from a proximal end of the delivery device to leverage the target tissue. In some aspects, in addition, the lever component may include a lever tool extending through the stabilizer passage; and the lever may include a support elevator associated with a passage through the stabilizer and configured to support and leverage said lever tool, the support elevator comprising one of: a flapper; a support and lever controller coupled to said support; a working channel; or a secondary inflatable balloon.

In some aspects, the stabilizer is an expandable stabilizer; the lever includes a tensioner extending between the expandable stabilizer and the target tissue; and expansion of the expandable stabilizer leverages the lever and the target tissue.

In some aspects, the stabilization and leverage device further includes a stabilizer controller associated with the stabilizer to facilitate manipulation of the stabilizer from a proximal end of the delivery device; and the lever comprises at least one of: a tether having a first end coupled to the target tissue and a second end coupled to the stabilizer controller; a tether extending from a first end coupled to the target tissue, through the stabilizer controller, and to a second end accessible at a proximal end of the delivery device for manipulation of the tether to leverage the target tissue; or a tether extending from a first end coupled to the target tissue, through a direction-changing structure on the stabilizer controller, and along the stabilizer controller to a second end accessible at a proximal end of the delivery device for manipulation of the tether to leverage the target tissue.

In some aspects, the stabilizer has a delivery device interface shaped and configured to receive a distal end of the delivery device during deployment of the stabilization and leverage device with the delivery device to a target tissue site. In addition, in some aspects, the lever may comprise a lever component loaded through a working channel of the delivery device and preloaded on a proximal end of the stabilizer mounted on a distal end of the delivery device. In addition or alternatively, in some aspects, the stabilizer has a ring opening therethrough shaped and configured to permit passage of an instrument therethrough.

In another aspect, a stabilization and leverage system is disclosed herein configured for delivery to a target tissue site. In some embodiments, the system is configured for stabilization of the target tissue site, and leveraging of target tissue at the target tissue site. In some aspects, the stabilization and leverage system includes a delivery device; a stabilizer configured for mounting on the delivery device; and a lever associated with the stabilizer and extending between the target tissue and the stabilizer to leverage the target tissue. The stabilizer stabilizes the target tissue independently of the distal end of the delivery device to allow independent free movement of the delivery device and instruments extended therethrough relative to the stabilizer and the target tissue

In some aspects, the stabilizer includes a stabilizer passage associated therewith; and the lever includes a lever component passing from a proximal end of the delivery device and through the stabilizer passage to be coupled to target tissue distal to the delivery device to leverage the target tissue. In addition or alternatively, in some aspects the lever includes a support elevator associated with the stabilizer passage; the stabilizer passage is configured for passage of a tensioner therethrough for coupling with the target tissue; and the support elevator and the stabilizer passage leverage the tensioner to leverage the target tissue. In addition or alternatively, in some aspects, the lever component includes a lever tool extending through the stabilizer passage; the lever includes a support elevator associated with a passage through the stabilizer; the support elevator comprises one of: a flapper; a support and lever controller coupled to said support; a working channel; or a secondary inflatable balloon; and the support elevator is configured to support and leverage the lever tool.

In some aspects, the stabilizer is an expandable stabilizer with a stabilizer passage associated therewith; the lever includes a tensioner extending between the expandable stabilizer and the target tissue; and expansion of the expandable stabilizer leverages the lever and the target tissue.

In some aspects, said stabilization and leverage device further includes a stabilizer controller associated with the stabilizer to facilitate manipulation of the stabilizer from a proximal end of the delivery device; and the lever comprises at least one of: a tether having a first end coupled to the target tissue and a second end coupled to the stabilizer controller; a tether extending from a first end coupled to the target tissue, through the stabilizer controller, and to a second end accessible at a proximal end of the delivery device for manipulation of the tether to leverage the target tissue; or a tether extending from a first end coupled to the target tissue, through a direction-changing structure on the stabilizer controller, and along the stabilizer controller to a second end accessible at a proximal end of the delivery device for manipulation of the tether to leverage the target tissue.

In some aspects, the stabilizer has a delivery device interface shaped and configured to receive a distal end of the delivery device during deployment of the stabilization and leverage device with the delivery device to a target tissue site. In addition or alternatively, in some aspects, the lever comprises a lever component loaded through a working channel of the delivery device and preloaded on a proximal end of the stabilizer mounted on a distal end of the delivery device. In addition or alternatively, the stabilizer has a ring opening therethrough shaped and configured to permit passage of an instrument therethrough.

In another aspect, a method of stabilizing a target tissue site and leveraging target tissue at the target tissue site is disclosed herein. In some aspects, the method comprises delivering, with a delivery device, a stabilization and leverage device loaded over the delivery device; deploying the delivery device to stabilize the target tissue independently of the distal end of the delivery device to allow independent free movement of the delivery device and instruments extended therethrough relative to the stabilizer and the target tissue; and extending a lever between the target tissue and the stabilizer and associating the lever with the stabilizer to leverage the target tissue.

In some aspects, the method further comprises deploying the stabilizer proximal to the target tissue site and proximal to the distal end of the delivery device; and extending the lever proximally from the target tissue site to the stabilizer and proximal to the distal end of the delivery device.

In some aspects, the method further comprises controlling the stabilizer via stabilizer controllers extending from the stabilizer to a proximal end of the delivery device; deploying the stabilizer distal to the target tissue site and distal to the distal end of the delivery device; extending the lever proximally from the target tissue site to the stabilizer controllers; and leveraging the lever with the stabilizer controllers to leverage the target tissue.

These and other features and advantages of the present disclosure will be readily apparent from the following detailed description, the scope of the claimed invention being set out in the appended claims.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a stabilization and leverage device in accordance with aspects of the present disclosure.

FIG. 2 illustrates a stabilization and leverage device in accordance with aspects of the present disclosure, showing an alternative lever to the one illustrated in FIG. 1.

FIG. 3 illustrates a stabilization and leverage device in accordance with aspects of the present disclosure, showing an alternative lever to the ones illustrated in FIGS. 1 and 2.

FIGS. 4 and 5 illustrate a stabilization and leverage device in accordance with aspects of the present disclosure, showing an alternative lever to the ones illustrated in FIGS. 1-3.

FIGS. 6 and 7 illustrate a stabilization and leverage device in accordance with aspects of the present disclosure, showing an alternative lever to the ones illustrated in FIGS. 1-5.

FIGS. 8 and 9 illustrate another stabilization and leverage device in accordance with aspects of the present disclosure.

FIGS. 10 and 11 illustrate a stabilization and leverage device in accordance with aspects of the present disclosure, showing an alternative stabilizer to the one illustrated in FIGS. 8 and 9.

FIGS. 12 and 13 illustrate another stabilization and leverage device in accordance with aspects of the present disclosure.

FIGS. 14 and 15 illustrate a stabilization and leverage device in accordance with aspects of the present disclosure, showing an alternative stabilizer to the one illustrated in FIGS. 12 and 13.

FIG. 16 illustrates another stabilization and leverage device in accordance with aspects of the present disclosure.

FIG. 17 schematically illustrates movement of the delivery device independent of the stabilization and leverage device of FIG. 16.

FIG. 18 illustrates a stabilization and leverage device such as in FIGS. 16 and 17, further illustrating a leverage device in accordance with some aspects of the present disclosure.

FIG. 19 illustrates a stabilization and leverage device in accordance with aspects of the present disclosure, showing an alternative lever to the one illustrated in FIG. 18.

FIG. 20 illustrates a stabilization and leverage device in accordance with aspects of the present disclosure, showing an alternative lever to the ones illustrated in FIGS. 18 and 19.

FIG. 21 illustrates another stabilization and leverage device in accordance with aspects of the present disclosure.

FIG. 22 illustrates a stabilization and leverage device as in FIG. 21, with the stabilizer and lever advanced for positioning to stabilize and leverage tissue.

FIG. 23 illustrates a stabilization and leverage device similar to that of FIGS. 21 and 22, but with a modified stabilizer shown in cross-section.

FIG. 24 illustrates a stabilization and leverage device as in FIG. 23, with the stabilizer and lever advanced positioned to stabilize and leverage tissue.

### DETAILED DESCRIPTION

The following detailed description should be read with reference to the drawings, which depict illustrative embodiments. It will be appreciated that the present disclosure is set forth in various levels of detail in this application. In certain instances, details that are not necessary for one of ordinary skill in the art to understand the disclosure, or that render other details difficult to perceive may have been omitted. All of the devices and/or methods disclosed and claimed herein can be made and executed without undue experimentation in light of the present disclosure. It should be understood that the claimed subject matter is not necessarily limited to the particular embodiments or arrangements described or illustrated herein, the scope of the claimed invention being set out in the appended claims.

As used herein, “proximal end” refers to the end of a device or object that lies closest to the user (medical professional or clinician or technician or operator or physician, etc., such terms being used interchangeably without intent to limit or otherwise), etc., such as when using the device (e.g., introducing the device into a patient, or during implantation, positioning, or delivery), and “distal end” refers to the end of a device or object that lies furthest from the user, such as when using the device (e.g., introducing the device into a patient, or during implantation, positioning, or delivery). A central axis means, with respect to an opening, a line that at least generally bisects a center point of the opening, extending longitudinally along the length of the opening when the opening comprises, for example, a tubular frame, a strut, or a bore.

As used herein, the singular forms “a”, “an”, and “the” include plural referents unless the content clearly dictates otherwise. As used in this specification and the appended claims, the term “or” is generally employed in its sense including “and/or” unless the content clearly dictates otherwise.

Various embodiments of a stabilization and leverage device, system, and method will now be described. Reference in this specification to “one embodiment,” “an embodiment,” “some embodiments”, “other embodiments”, etc. indicates that one or more particular features, structures, and/or characteristics in accordance with principles of the present disclosure may be included in connection with the embodiment. However, such references do not necessarily mean that all embodiments include the particular features, structures, and/or characteristics, or that an embodiment includes all features, structures, and/or characteristics. Some embodiments may include one or more such features, structures, and/or characteristics, in various combinations thereof. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. When particular features, structures, and/or characteristics are described in connection with one embodiment, it should be understood that such features, structures, and/or characteristics may also be used in connection with other embodiments whether or not explicitly described, unless clearly stated to the contrary. Moreover, various features are described which may be exhibited by some embodiments and not by others. Similarly, various features or requirements are described which may be features or requirements for some embodiments but may not be features or requirements for other embodiments

In accordance with principles of the present disclosure, a stabilization and leverage device or tool (such terms being used interchangeably without intent to limit or otherwise convey different meaning or intent) includes a stabilizer and a lever. The stabilization and leverage device may be used in conjunction with a delivery device, such as to be delivered and deployed at a target tissue site. The stabilizer and lever may be incorporated into a tissue stabilizing and leveraging system. Such system may incorporate other devices, tools, instruments, components, structures, etc. (such terms used interchangeably herein for the sake of convenience and without intent to limit) known to those of ordinary skill in the art or heretofore developed to perform an operation or procedure or the like (hereinafter “procedure” for the sake of convenience without intent to limit) on a body part, such as an organ, or vessel, or tissue. The stabilization and leverage device is described herein in conjunction with a procedure performed on body tissue. However, it will be appreciated that the procedure need not be so limited. Moreover, the method of stabilizing and leveraging disclosed herein, such as to be performed in association or conjunction with the device and system disclosed herein, is described in conjunction with a procedure performed on body tissue, but need not be so limited.

In embodiments formed in accordance with principles of the present disclosure, the delivery device is a known device or structure configured to carry and deliver the stabilizer and the lever through a body passage to the target (e.g., target tissue site) where the procedure is to be performed. The delivery device may be a tube, endoscope, guide lumen, working channel, sheath, port(s), access port(s), catheter, passage, etc. In some embodiments, the delivery device is flexible and/or articulated to permit navigation through a non-straight or tortuous body passage or lumen. The delivery device may include an overtube or other additional passageway connected in any way known in the art or heretofore known, including, without limitation, being separate and running alongside, or being integral with the delivery device. The overtube may be configured for passage and/or delivery of another element of the device or system therethrough. The overtube and delivery device may be made of the same or different materials. It will be appreciated that stabilizers and associated components disclosed herein may be attached to a scope or combined with an overtube that the scope would travel through and move freely in and out of.

The stabilizer is shaped and configured to contact the body part (e.g., tissue) adjacent or surrounding the target of the procedure (e.g., target tissue site), and to stabilize the environment in which the procedure is to be performed, such as relative to the working tools (e.g., medical instruments, endoscope, and/or other devices). For instance, if the procedure is to be performed in the gastrointestinal (GI) tract, the stabilizer is shaped and configured to contact GI tissue to stabilize the target tissue site such as so that autonomic movements (e.g., peristalsis) do not drive movement of the tissue or otherwise have an effect on the tissue so that the procedure can be performed without interference or interruption as desired. For instance, the stabilizer may be shaped and configured to hold in place and/or stretch the tissue at or adjacent or surrounding (used interchangeably herein without intent to limit) the target tissue site. Examples of stabilizers are expandable members (e.g., a balloon, stent, self-expanding structure such as mesh or coil structure, expandable structure responsive to expand upon application of force thereto, or the like), a wire petal, a wire bulb, etc., the principles of the present disclosure not necessarily limited to the specific structure.

The stabilizer is preferably mounted on, loaded over, or otherwise attachable to the delivery device to be delivered to the target site with the delivery device, the delivery device being configured to facilitate delivery of other devices, instruments, tools, etc. (such terms being used interchangeably herein without intent to limit) to the target site. As such, the stabilizer and delivery device may be considered together to be a stabilization system.

A target tissue fastener/anchor/clip or other tissue-engagement structure (“fastener” used herein for the sake of convenience without intent to limit or otherwise convey different meaning or intent) typically is used to couple or attach or connect to, or grasp, or the like, the target tissue. The target tissue fastener typically has structure shaped and configured to engage/grasp/pinch/hold/couple to/fasten to/loop around (such terms being used interchangeably herein without intent to limit or otherwise convey different meaning or intent) tissue and may be any of the following or equivalents thereof, without limitation: fastener, clip, suture, corkscrew, barb, spike, hook, helix, grasper, engagement member, staple, adhesive, loop (simple, spiral, helical, etc.), cloth, wire, elastic, etc. Typically, the target tissue fastener is shaped to engage, grasp, be coupled to (e.g., looped around, enclosed around, etc.), etc., target tissue such as with a grasper or grasping device or other manipulation tool as known in the art or heretofore known. The target tissue fastener may be repositionable or not, depending on the needs or circumstances or otherwise. The target tissue fastener may optionally be a part of a tissue traction device configured for coupling to target tissue and another element. For instance, a tissue traction device may include a tissue fastener (coupled to the target tissue), a tether, and an anchoring or stabilizing fastener (coupled to another site, such as another tissue site, to stabilize and/or leverage the target tissue fastener).

As discussed above, it is generally desirable to apply traction to the target tissue fastener to apply traction to and to leverage the target tissue. In accordance with principles of the present disclosure, a lever is provided in conjunction with a tissue stabilizer for use by a clinician to leverage the target tissue anchor to apply traction or tension to the target tissue (e.g., via a target tissue fastener) or otherwise to manipulate the target tissue fastener and the target tissue to facilitate performance of a procedure thereon or therearound (e.g., put the target tissue under tension to facilitate performance of a procedure thereon—such as by retracting the tissue to facilitate cutting of the tissue; facilitate access of the delivery device, scope, tool, etc. to the target tissue site; provide sufficient room or clearance for tools to be manipulated or otherwise operated to perform the desired procedure; and/or to facilitate visualization of the target tissue site). The lever is preferably provided in conjunction with the stabilizer, e.g., positioned with respect to the stabilizer, and preferably associated with (e.g., coupled to) the stabilizer. The stabilizer and lever may be considered together a stabilization and leverage device. In some embodiments, the lever is coupled to, mounted to or on, or otherwise associated with the stabilizer. In some embodiments such association is direct. In other embodiments, such association is via another element or component of the stabilization and leverage device. The stabilizer, lever, and delivery device may be considered together to be a stabilization and leverage system.

The lever includes one or more components arranged, configured, shaped, positioned, etc., to interact with target tissue (e.g., be coupled with the target tissue, such as via a tissue fastener) to apply leverage thereto to apply tension or traction to the target tissue. In some embodiments, the lever may include or be associated with a target tissue fastener or with a tissue traction device to interact with the target tissue. The lever may include one or more of the following components: a lever tensioner or tension mechanism or lever controller (such terms used interchangeably herein without intent to limit or otherwise convey different meaning or intent); a lever tool; a support; a direction-changing structure; a tissue traction device. The lever tensioner applies tension or traction or otherwise leverages a further component of the lever and/or the target tissue. In some embodiments the lever tensioner is flexible. In some embodiments, the lever tensioner is a structure such as a cable, tether, cord, suture, wire, string, dental floss, rod, tube, spring, elastic band, thread, cloth, or the like via which tension may be applied or by which another component of the lever, or the tissue fastener, may be controlled. In some embodiments the lever tensioner is elastic. In some embodiments, the lever tool is a grasper or other tool which may engage, manipulate, or otherwise, the target tissue, either directly or via another component (e.g., via a tissue fastener or a tether). In some embodiments, the support is shaped and configured to support another component, such as a lever tool, or to support and guide another component such as a tensioner. In some embodiments, the direction-changing structure is a pivot or passageway or other structure shaped and configured such that another lever component, or otherwise, which interacts therewith changes direction or is redirected such as for leveraging purposes. In some embodiments, the direction-changing structure is a hook, loop, pulley system, channel, etc. In some embodiments, the tissue traction device is a tissue-engaging traction device, such as known in the art, and includes components configured to engage the target tissue as well as to apply traction to the target tissue. One example of a tissue traction device is a tether traction device which has a tether (generally elastic) with a tissue fastener on one end and a fastener such as a stabilization anchor or fastener (by which the tether traction device is anchored so that the tether applies traction to the tissue fastener and tissue grasped by the tissue fastener) on the other end. In general, various components of the lever may be configured and/or shaped and/or formed of a material that facilitates movements to apply tension or traction to the target tissue

Returning to discussion of use of a target tissue fastener or a tissue traction device, it will be appreciated that it may be desirable to provide a stabilizing anchor to stabilize the target tissue fastener and/or tissue traction device and/or any tensioner applying tension or traction to the target tissue fastener so that the stability of the target tissue is not affected by the delivery device or the performance of the procedure on the target tissue (e.g., not affected by movement of medical instruments or the like in the region of or directly on the target tissue). In some embodiments of the present disclosure, the stabilizing anchor may be associated with the stabilizer. The target tissue anchor, the stabilizing anchor, and the tensioner may be considered, in combination, a tissue retraction mechanism.

In accordance with principles of the present invention, in use, the stabilization and leverage device leverages the target tissue for various benefits, such as to allow the scope and any instruments or tool (such as within the scope's channel) to gain access to and visualization of the target tissue, and/or to put tissue under tension to facilitate manipulation of the instrument or tool relative to a target tissue (such as to perform a procedure on the target tissue), and/or to lift or retract or otherwise manipulate the target tissue while performing a procedure on (e.g., cutting underneath) the target tissue or the like. If a resection or dissection is performed on the target tissue, once the target tissue has been fully resected or dissected, the target tissue can remain with the fastener and be removed with the delivery device. The stabilizing anchor may be removed (e.g., with a component of the stabilization and leverage device to which it is coupled) or left in place (e.g., if anchored to another tissue) to naturally slough off.

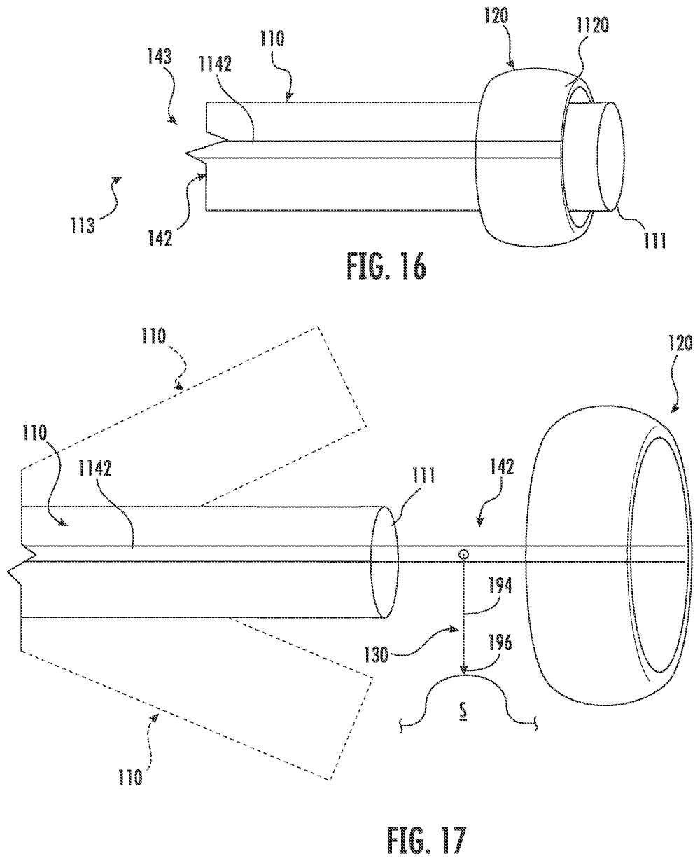

Turning now to the Figures, various embodiments of stabilization and leverage devices combining various embodiments of a delivery device, a stabilizer, and a lever, and optionally additional embodiments of additional components, devices, instruments, tools, etc., are illustrated for the sake of disclosing and describing informative examples without intent to limit the disclosure from the broad principles described herein. FIGS. 1-24 illustrate embodiments of a stabilization and leverage device 100 formed in accordance with various principles of the present disclosure, with a stabilizer 120, and a lever 130 associated with the stabilizer 120 such as by being coupled thereto (yet, optionally, spaced therefrom) or mounted thereon. In some embodiments the lever 130 is coupled directly to the stabilizer 120. In some embodiments, the lever 130 is coupled to the stabilizer 120 via another component. For instance, in some embodiments, the lever 130 is coupled to the stabilizer 120 via a component which also may be used to control and/or manipulate the stabilizer 120. In the embodiments of FIGS. 1-15, the stabilizer 120 is associated with a delivery device 110 at a location proximal to the distal end 111 of the delivery device. In the embodiments of FIGS. 16-24, the stabilizer 120 is delivered with a delivery device 110 to the target tissue site T and then moved distally to be positioned distal to the distal end 111 of the delivery device 110 during a procedure to be performed on the target tissue T.

In some embodiments, the stabilizer 120 of a stabilization and leverage device 100 formed in accordance with principles of the present disclosure may be in the form of an expandable stabilizer, the expansion of which applies pressure or tension or the like to stabilize the target tissue T, and which also provides the leverage to the lever 130 and or other tensioner and/or fastener and/or tool leveraging the target tissue T. The expandable stabilizer preferably is in a retracted or compact configuration while navigated with the delivery device 110 to the target tissue site S. The stabilizer 120 is deployed, such as inflated or expanded, or otherwise configured and positioned to engage the body site at, adjacent, in the vicinity of, etc., (such terms used interchangeably herein without intent to limit) the target tissue site S to stabilize such body site (e.g., tissue) to facilitate performance of the desired procedure at the target tissue site S (e.g., on the target tissue T). In some embodiments, the stabilizer 120 is in the form of an inflatable stabilizer such as an inflatable balloon, inflated, for instance, via any inflation/deflation mechanism known or heretofore known in the art (such mechanism not being critical to the principles of stabilization and leverage of the present disclosure), such as inflation tubes or lumens or the like. In some embodiments, control tubes or deployment tubes or inflation tubes/lumens, or the like, are in fluid communication with the inflatable stabilizer and fluid (e.g., air or water) is passed therethrough to inflate the inflatable stabilizer, or suction is applied therethrough to deflate the inflatable stabilizer. In some embodiments, the stabilizer 120 is in the form of an expandable stent stabilizer, which may be deployed in a compressed configuration, and expanded when desired, such as by manipulation of the stent stabilizer to release a restraint thereon (maintaining the stent stabilizer in the compressed configuration). In some embodiments, a controller, such as a cable or wire or other stent operator or controller known in the art or heretofore known (such element not being critical to the principles of stabilization and leverage of the present disclosure), is manipulated (e.g., from a proximal end thereof) to manipulate the stent stabilizer to expand or compress.

The delivery device 110 illustrated in the figures herein is an endoscope with a delivery lumen or passage or working channel 112 (“working channel” generally used herein for the sake of convenience without intent to limit) through which a working tool 180 (e.g., a cutting tool) may be transported to a delivery site/target tissue site S any time during the procedure. However, it will be appreciated that other forms of delivery devices 110 are within the scope of the present disclosure, the current embodiment only showing an endoscope for convenience of illustration. As illustrated, the stabilizer 120 is mounted on (e.g., loaded over or positioned over) a portion of the delivery device 110 (e.g., coupled to, e.g., and exterior, of the delivery device 110, such as by passing the delivery device 110 through a passage 124 through the stabilizer 120).

The stabilizer 120 of the embodiments of FIGS. 1-7 has a passage 124 through which a lever tool, device, or instrument 190 (used alternately, in conjunction with the term “lever” without intent to limit, it being appreciated that the tool, device, instrument, etc., is not critical to the stabilization and leverage principles disclosed herein), such as a grasper, is passed to reach and to manipulate and to leverage the target tissue T. It will be appreciated that in the embodiments of FIGS. 1-7, the lever tool 190 may be a tensioner and fastener which directly grasps, engages, fastens to, etc. (such terms being used alternately without intent to limit), the target tissue T (such as illustrated in FIG. 1), or a combination of a grasper or clip and a separate tensioner/fastener (such as illustrated, for example, in FIG. 5). A lever 130 is associated with the stabilizer 120 to leverage the lever tool 190, which may be considered a component of the lever 130. In the various embodiments of FIGS. 1-7, the lever 130 includes a support elevator 132 configured with respect to the stabilizer 120 to raise the lever tool 190 to leverage the target tissue T. The elevator 132 may be in any of a variety of different forms or configurations, several examples of which will now be described. The lever 130 may include a lever controller 134 via which the lever 130 and/or a component thereof (e.g., the elevator 132) may be operated or manipulated or navigated, such terms being used interchangeably herein without intent to limit.

The example of an elevator 132 illustrated in FIG. 1 is in the form of a wedge or flipper or other movable (e.g., pivotable) support elevator 232 supporting or otherwise engaged with the lever tool 190 so that movement of the support elevator 232 causes the desired movement of (e.g., elevated, raised, or otherwise moved, such as laterally) the lever tool 190 to leverage the target tissue T as desired. Movement or control of the support elevator 232 may be achieved via manipulation of a controller 234 (e.g., a balloon, or a cable, tether, cord, suture, controller, or the like, such terms being used interchangeably herein without intent to limit, it being understood that the controller for the support is not critical to the stabilization and leverage principles disclosed herein) at a proximal end 115 (e.g., at the handle) of delivery device 110.

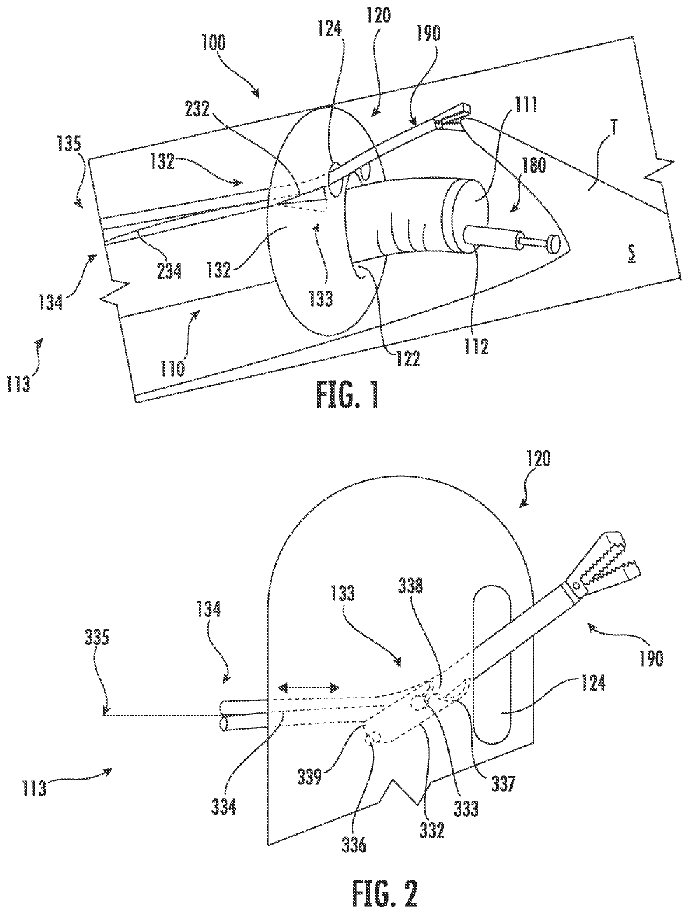

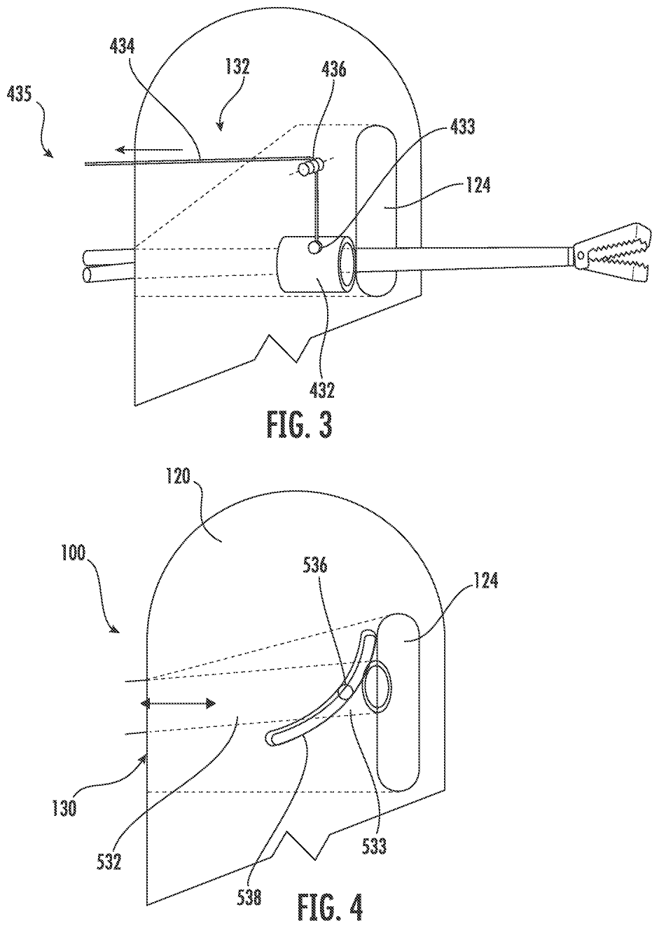

The example of an elevator 132 illustrated in FIGS. 2 and 3 is in the form of a support channel 332, 432 supporting or coupled or engaged with the lever tool 190 so that movement of the support channel 332, 432 (e.g., an open-sided channel 332, as in FIG. 2, or a fully enclosed channel 432, as in FIG. 3) causes the desired movement of (e.g., can be elevated, raised, or otherwise moved, such as laterally) the lever tool 190 to leverage the target tissue T as desired. Movement or control of the support channel 332, 432 may be achieved via manipulation of a lever controller 334, 434 (e.g., a cable, tether, cord, suture, controller, or the like, such terms being used interchangeably without intent to limit, it being understood that the controller is not critical to the stabilization and leverage principles disclosed herein) at a proximal end 113 (e.g., at the handle) of the delivery device 110. For instance, the distal end 333, 433 of the controller 334, 434 may be coupled to the support channel 332, 432 and the proximal end 335, 435 of the controller 334, 434 may be accessible to be manipulated at the proximal end 115 of the delivery device 110.

In the embodiment of FIG. 2, the support channel 332 is an open-sided channel supporting the lever tool 190 from below. The support channel 332 is mounted within the passage 124 in the stabilizer 120, via a direction-changing structure (e.g., pivotably mounted via a pivot pin 336) at one end of the support channel 332 (e.g., a proximal end 339), and the distal end 333 of the controller 334 is coupled to a distal end 337 of the support channel 332 (such as to an exterior or end of the support channel 332 such as via a hook or slit or other anchoring configuration 338). Pulling on the proximal end 335 of the controller 334 causes the distal end 333 of the controller 334 to pivot, leverage, or otherwise move the support channel 332 to move or leverage the lever tool 190 and thereby to leverage the tissue grasped or otherwise engaged by the lever tool 190.

In the embodiment of FIG. 3, the support channel 432 is a substantially closed channel enclosing the lever tool 190 to support the lever tool 190. The support channel 430 is freely movable within the passage 124 in the stabilizer 120, to be moved (e.g., elevated) by the controller 134 which, in turn, can be leveraged within the passage 124 such as by passing about direction-changing structure 436 such as a lever point or pivot or pulley or the like. In the illustrated example, the controller 134 is in the form of a tensioner 434 (e.g., cable, tether, cord, suture, controller, or the like) coupled at one end to the support channel 432 (such as to an exterior surface of the support channel 432 via any known connection in the art) and extended about a direction changing structure 436 within the passage 124 in the stabilizer 120. The direction changing structure 436 may be a pivot pin or other structure about which the controller 134 may be passed so that movement of the controller 134 leverages (e.g., lifts or lowers) the support channel 430 to thereby leverage the lever tool 190 and the target tissue T associated or coupled therewith. In use, pulling on the proximal end 435 of the controller 434 causes the distal end 433 of the controller 434 to pivot, leverage, or otherwise move the support channel 430 within the passage 124 in the stabilizer 120 to move or leverage the lever tool 190 and thereby to leverage the tissue grasped or otherwise engaged by the lever tool 190.

In the embodiment of FIGS. 4 and 5, the lever tool 190 passes through and is supported and moved by a lever 130 including a working channel 532. The working channel 532 passes through the passage 124 in the stabilizer 120. In some embodiments, the working channel 532 may be freely movable within the passage 124 to move (e.g., elevate) and leverage the lever tool 190 and the target tissue engaged by the lever tool 190. In one embodiment, the working channel 532 extends the full length of the delivery device 110 and is movable (e.g., may be pulled or pushed) from a proximal end 115 of the delivery device 110 to move the distal end 533 of the working channel 532, thereby leveraging the lever tool 190. In one embodiment, the working channel 532 may engage the stabilizer 120, such as via an internal wall of passage 124, to facilitate or guide movement of the working channel 532 within the passage 124. In one embodiment, a lever engagement of the working channel 532 and the stabilizer 120 may be provided, such as a pivot or pulley or other direction-changing structure. For instance, in the embodiment illustrated in FIGS. 4 and 5, a lever engagement is in the form of a projection or pin 536 on one of the working channel 532 and the passage 124 and a slot 538 in the other of the working channel 532 and the passage 124. For instance, the slot 538 may be in the form of a curved slot or channel or the like, shaped and configured to engage and/or receive the pin 536 to allow relative movement therebetween, resulting in movement of the working channel 532 and consequent movement of the lever tool 190 to leverage the target tissue T. In one embodiment, such as illustrated as an example in FIGS. 4 and 5, in use, the working channel 532 is moved distally so that a pin 536 projecting from a side thereof moves upwardly along a distally upwardly extending slot 538. The lever tool 190 is thereby moved distally out of the delivery device 110 and levered upwards to apply upward leverage to the target tissue T (which may be adjacent a lower side of stabilization and leverage device 100) such as via a separate tissue traction device 192 such as known in the art. For instance, the tissue traction device 192 may include a tether 194 with a tissue fastener 196, 198 at each end thereof. It will be appreciated that the tissue fastener is shown schematically throughout the drawings and encompasses tissue fasteners known in the art and heretofore known, the present disclosure not being limited by a particular tissue fastener configuration.

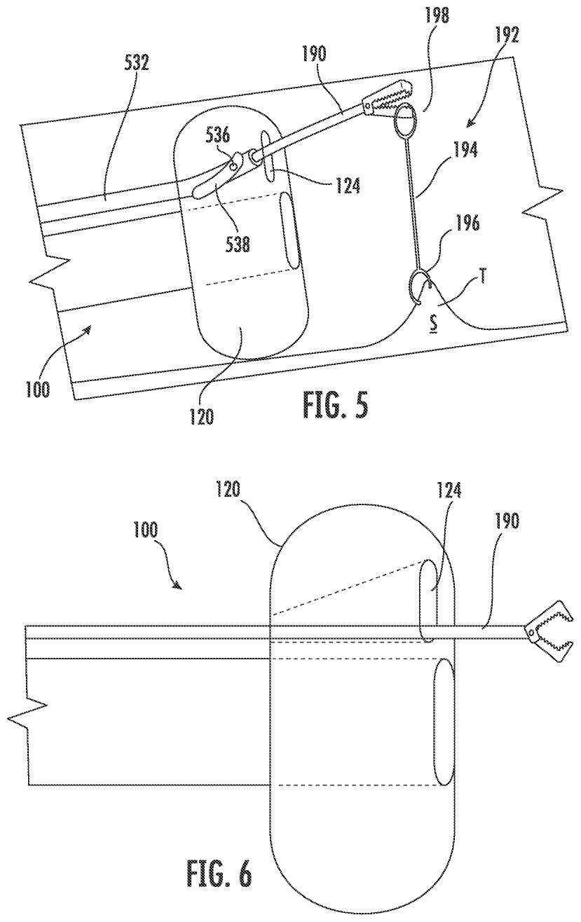

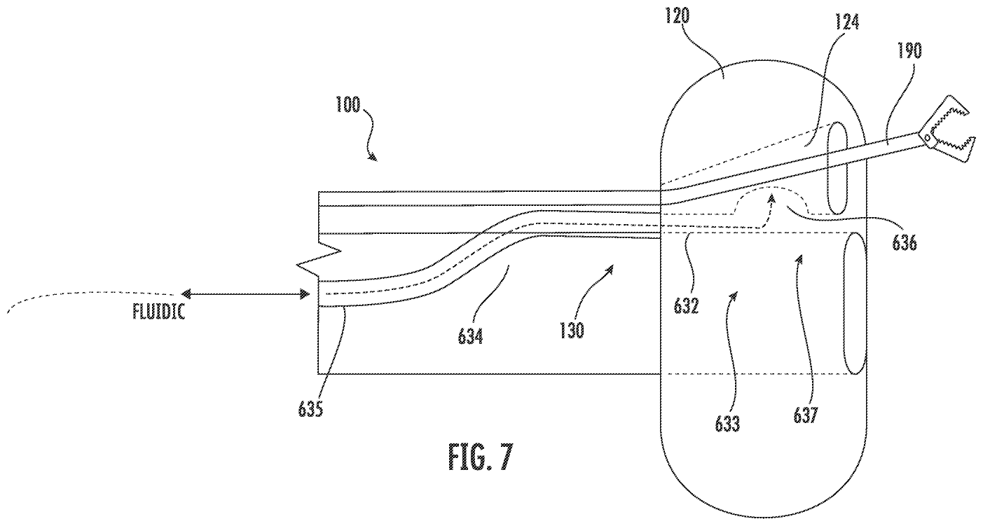

In the embodiment of FIGS. 6 and 7, the lever tool 190 is supported and moved by a lever 130 including a secondary fluidic balloon 632. In one embodiment, the secondary fluidic balloon 632 has a lumen 634 (e.g., an inflation tube or hose or the like) extending the full length of the delivery device 110, with a balloon 636 at a distal end 633 of the lumen 634. The distal end 637 of the secondary fluidic balloon 632 passes through the passage 124 in the stabilizer 120 so that the secondary fluidic balloon 632 is positioned within the passage 124. A fluid (e.g., air or water) may be passed through a proximal end 635 of the lumen 631 to fill the secondary fluidic balloon 632. As the secondary fluidic balloon 632 fills, it inflates and acts as an elevator to elevate the lever tool 190, thereby leveraging the lever tool 190 and the target tissue T coupled thereto. In one embodiment, the secondary fluidic balloon 632 is inserted through a separate balloon channel in the delivery device 110. In one embodiment, the secondary fluidic balloon 632 is mounted in a separate working channel of the delivery device 110, such as through a deployment catheter (as known in the art, and thus not illustrated, in the interest of simplifying the drawings).

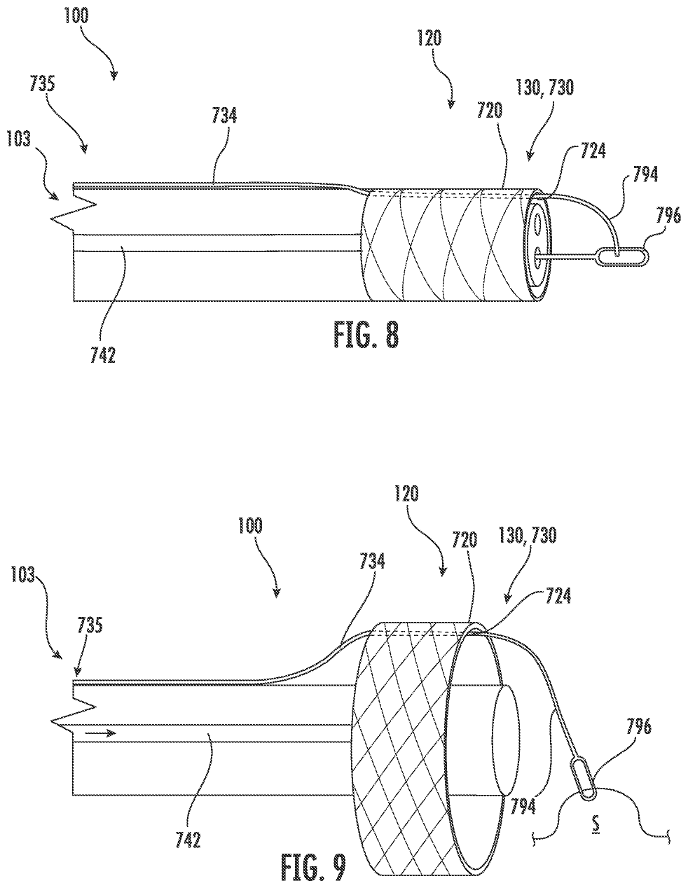

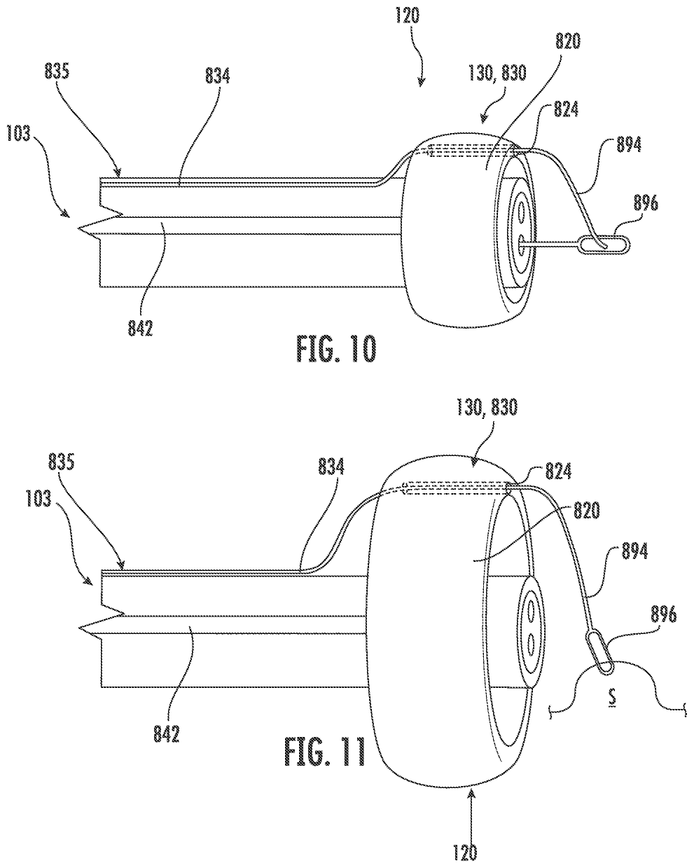

In some embodiments, such as those illustrated in FIGS. 8-11, the expansion of an expandable stabilizer 120 may provide tissue stabilization as well as leverage to a component of a lever 130 of a stabilization and leverage device 100. In the embodiment of FIGS. 8 and 9, the expandable stabilizer in the form of an expandable stent stabilizer 720, and in the embodiment of FIGS. 10 and 11, the expandable stabilizer is in the form of an inflatable balloon stabilizer 820. Expansion of the expandable stabilizer 720, 820 (such as via a stabilizer controller 742, 842, such as a control wire or inflation tube, respectively) brings the expandable stabilizer 720, 820 to the desired size to engage the body site at, adjacent, in the vicinity of, etc., the target tissue site S to stabilize the target tissue site S in accordance with principles of the present disclosure. A lever 130 extends through a channel or passage 724, 824 (such terms being used interchangeably, without intent to limit) associated with (e.g., extending along, such as in/through or over) the expandable stabilizer 720, 820 to be leveraged by the expandable stabilizer 720, 820, such as upon expansion of the expandable stabilizer 720, 820, to apply desired leverage to the target tissue T. More particularly, in one embodiment, the lever 730, 830 includes a lever tensioner 734, 834 extending through the stabilizer passage 724, 824 and coupled to the target tissue T, such as via a tissue fastener or tissue traction device, such as known or heretofore known in the art. In some embodiments, the lever 730, 830 may include a lever tool, such as described herein, coupled to a tissue fastener or tissue traction device. Adjustment (e.g., expansion, inflation, deflation, movement, etc.) of the expandable stabilizer 720, 820 leverages the lever tensioner 734, 834 passing through the passage 724, 824 through the stabilizer 720, 820, the passage 724, 824, with expansion of the stabilizer 720, 820, thus acting as a lever in this embodiment. It will be appreciated that such leveraging may be achieved whether or not the passage 724, 824 runs the entire length of the delivery device 110. In the illustrated embodiment, the passage 724, 824 is located at the top of the expandable stabilizer 720, 820, and the target tissue T is positioned on another side thereof (e.g., the bottom of the expandable stabilizer 720, 820) so that expansion of the expandable stabilizer 720, 820 lifts the lever tensioner 734, 834 to leverage the target tissue T, such as via the tissue fastener 796, 896 (shown schematically, with an optional tether 794, 894 extending between the tissue fastener 796, 896 and the lever tensioner 734, 834). The lever tensioner 734, 834 may be manipulated (e.g., pulled or otherwise moved), such as via a proximal end 735, 835 thereof (such as at the proximal end 115 of the delivery device 110), to apply further traction to the lever tensioner 734, 834 and the target tissue T. In the embodiment illustrated in FIGS. 8-11, running of the lever tensioner 734, 834 through the passage 724, 824 in the expandable stabilizer 720, 820 provides leverage so that when the lever tensioner 734, 834 is pulled at its proximal end 735, 835, it is pulled at an upward angle to facilitate access to the target tissue T, such as by allowing access for a cutting tool (e.g., and ESD knife) to cut underneath the target tissue T (e.g., a lesion).

The tensioner/controller 734, 834 may be in the form of a typical tensioner or controller used in conjunction with a target tissue fastener 196 to apply tension or leverage thereto to lift or leverage the target tissue T to facilitate performance of a procedure on the target tissue T. Alternatively, the tensioner/controller 734, 834 may be a separate controller coupled to a typical tensioner or controller coupled to a target tissue fastener 196. In either configuration (the disclosure not being limited to either configuration or alternate configurations which are not critical to the broader stabilization and leverage principles disclosed herein), the tensioner/controller 734, 834 extends through the passage 724, 824 to a proximal end 113 of the delivery device 110 where a proximal end 733 of the tensioner/controller 734, 834 can be manipulated to affect (e.g., increase, decrease, alter direction of, etc.) tension on the tensioner/controller 734, 834, thereby affecting tension on the target tissue fastener 196.

In use, optionally, a tissue fastener 796, 896 or tissue traction device or the like may be extended or deployed or delivered through a working channel 112 of the delivery device 110 of the embodiment illustrated in FIGS. 8-11 to the target tissue T. A tensioner 734, 834, such as in the form of a suture, wire, cable, tether, cord, or the like, is coupled to the target tissue fastener 196 and run through the passage 724, 824 through the expandable stabilizer 720, 820 alongside the delivery device 110 to the proximal end 113 of the delivery device 110. When the delivery device 110 reaches the target tissue site S, the expandable stabilizer 720, 820 is expanded, to provide stabilization, and the tissue fastener 796, 896 is deployed or otherwise attached to the target tissue T. When the expandable stabilizer 720, 820 is expanded, the lever tensioner 734, 834 is lifted therewith to leverage the tissue fastener 796, 896. When further leveraging or lifting of the target tissue T is desired or required, the lever tensioner 734, 834 is manipulated (e.g., pulled) at the proximal end 735 thereof (generally at the proximal end 113 of the delivery device 110) to provide the desired leverage, such as to lift the target tissue T to the desired point.

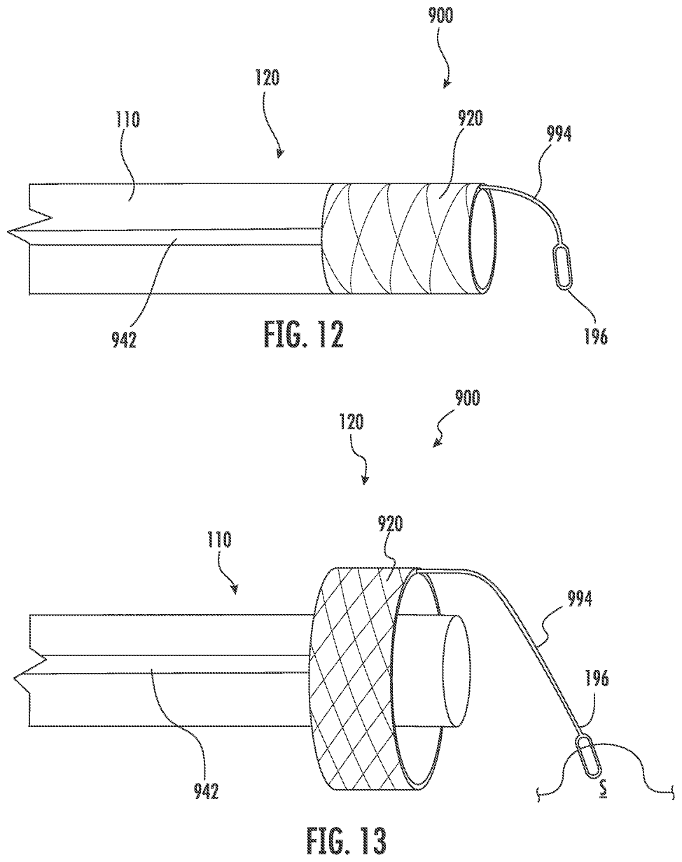

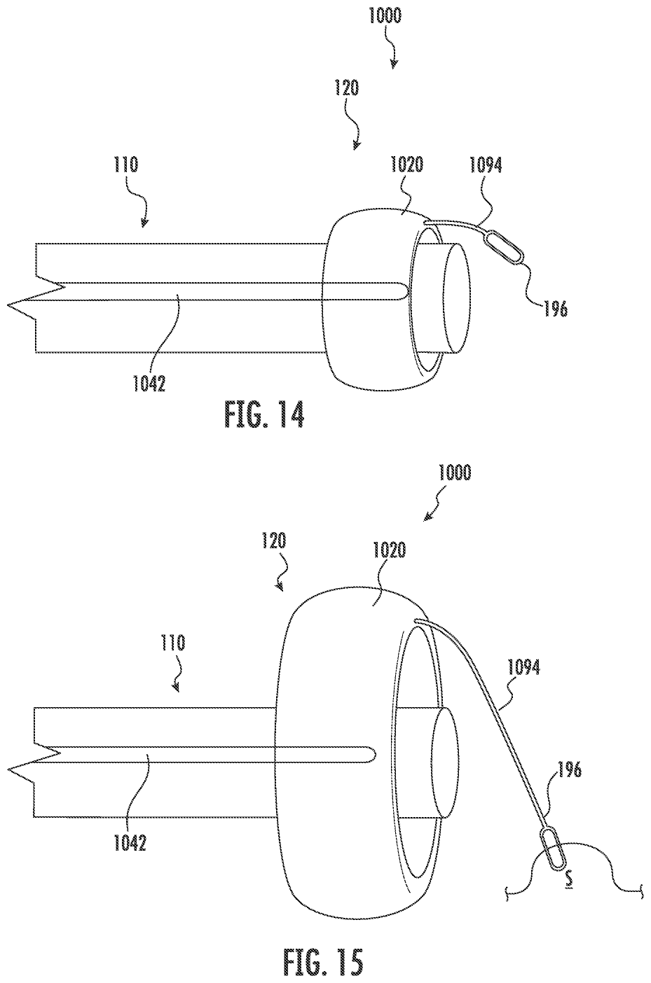

Similar leveraging of the target tissue T, as described with reference to FIGS. 8-11, such as via leveraging of a target tissue fastener 196 with an expandable stabilizer 120, may be accomplished with the embodiments of FIGS. 12-15, which similarly include an expandable stabilizer 120, such as in the form of an expandable stent stabilizer 920, illustrated in FIGS. 12 and 13, or an inflatable balloon stabilizer 1020, as illustrated in FIGS. 14 and 15. However, instead of the lever 130 including a tensioner extending through a passage through or associated with the expandable stabilizer 120, the lever 130 of the embodiment of FIGS. 12-15 includes a tether 994, 1094 coupled to the expandable stabilizer 920, 1020. For instance, in the embodiment of FIGS. 12 and 13, the tether 994 may be directly attached to, or woven into the material of the expandable stent stabilizer 920. In the embodiment of FIGS. 14 and 15, the tether 1094 may be coupled to, such as directly attached to, an inner or outer side of the inflatable balloon stabilizer 1020.

In one embodiment, the tether 994, 1094 may be an elastic material that is coupled to the expandable stabilizer 920, 1020 at one end and to a tissue fastener 196 at another end. The tissue fastener 196 (shown schematically) may be coupled to the target tissue T with the use of a tool, such as a grasper, inserted through the working channel 112 of the delivery device 110 (see, e.g., FIG. 1) to the target tissue site S. Movement of the expandable stabilizer 920, 1020 (such as upon expansion thereof, in a manner such as described in connection with the expandable stabilizers 720, 820 of the embodiment illustrated in FIGS. 8-11, such as via a stabilizer controller 942, 1042, such as a control wire or inflation tube, respectively) applies force or tension to the tether 994, 1094 to leverage the tissue fastener 196 attached thereto and thereby to leverage the target tissue T. When the target tissue T has been fully dissected, the lesion can either remain attached to the tissue fastener 196 and be removed with the delivery device 110, or a grasper can detach the tissue fastener 196 from the target tissue T. It will be appreciated that, as with the embodiments of FIGS. 8-11, the stabilization and leverage device 900, 1000 of FIGS. 12-15 similarly stabilizes tissue at or adjacent or surrounding the target tissue T upon expansion of the expandable stabilizer 920, 1020 thereof, and leverages the lever 130 to lever the target tissue T.

It will be appreciated that in the embodiments described thus far, the lever 130 is associated with the stabilizer 120, such as by being mounted on or formed with the stabilizer 120. In alternate embodiments of stabilization and leverage devices 100 formed in accordance with principles of the present disclosure, the lever 130 may be associated with the stabilizer 120 via another component of the stabilization and leverage device 100. For example, in the embodiments of FIGS. 16-20, the lever 130 is coupled to the stabilizer 120 via a stabilizer controller 142 coupled to the stabilizer 120 to deploy and/or to control and/or to navigate and/or to manipulate (used interchangeably herein without intent to limit) the stabilizer 120. For instance, in the embodiment of FIGS. 16-20, in which the illustrated stabilizer 120 is in the form of an inflatable stabilizer, the stabilizer controller 142 may be a stabilizer controller 1142, 1242, 1342 in the form of control tubes and/or inflation lumens or the like which may be used (e.g., pushed, pulled, moved laterally or up or down) not only to manipulate the position of the stabilizer 120, but also to control the stabilization imparted by the stabilizer 120 (e.g., by imparting fluidic control to the stabilizer 120 to control expansion and thus support or pressure applied to the target tissue site S to stabilizer the target tissue site S). One end (or component) of the lever 130 in the embodiments of FIGS. 16-20 is coupled to or supported by the stabilizer controller 1142, 1242, 1342, and the other end (or another component) is coupled to the target tissue T, such as via a tissue fastener 196 (shown schematically) or the like. In some embodiments the lever 130 includes a tether 194 with one end coupled with a tissue fastener 196 coupled to the target tissue T, and another end coupled with the stabilizer controller 142.

In use, the stabilizer 120 of the embodiments of FIGS. 16-20 may be loaded on the distal end of the delivery device 110, as shown in FIG. 16. In the illustrated embodiments of FIGS. 16-20, the stabilizer controller 142 is mounted over or runs alongside the delivery device 110, such that the stabilizer 120 is delivered with the delivery device 110 to the target tissue site S. In some embodiments, a rigid or semi-rigid attachment may be used to mount the stabilizer controller 1142, 1242, 1342 along the delivery device 110 to extend from a distal end 111 of the delivery device 110 to a proximal end 113 of the delivery device 110 to push the stabilizer 120 distally to the target tissue site S. Once the delivery device 110 and the stabilizer 1120, 1220, 1320 of the embodiment of FIGS. 16-20 are at the target tissue site S, the stabilizer 120 is moved distally beyond the distal end 111 of the delivery device 110 and distally beyond the target tissue site S. The stabilizer controller 1142, 1242, 1342 may be manipulated by manipulating a proximal end 1143, 1243, 1343 of the stabilizer controller 1142, 1242, 1342. Typically, the stabilizer 120 is deployed in an unexpanded (e.g., uninflated) configuration, and is expanded (e.g., inflated, if in the form of a balloon, via the stabilizer controller 1142, 1242, 1342 if in the form of an inflation tube) after being positioned past the target tissue site S to stabilize tissue at/slightly distal to the target tissue site S. Once the stabilizer 120 has been deployed to stabilize the target tissue site S, and the lever 130 is coupled to the target tissue T (e.g., via a tissue fastener and/or tissue traction device or the like) to leverage the target tissue T, further tools may be deployed to perform the desired procedure on the target tissue T or otherwise. It will be appreciated that the delivery device 110 and any further tools delivered or deployed via the delivery device 110 are movable independently of the stabilizer 120 such that the stabilizer 120 effectively stabilizes the target tissue site S in accordance with principles of the present disclosure. Independent movement of the delivery device 110 (relative to the stabilizer 120 and lever 130) is schematically illustrated (broken line illustrations of the delivery device 110 showing alternate positions of the delivery device 110) in FIG. 17. If desired, adjustment of the leverage is possible by manipulation of the position of stabilizer controllers 1142, 1242, 1342 and/or stabilizer 1120, 1220, 1320.

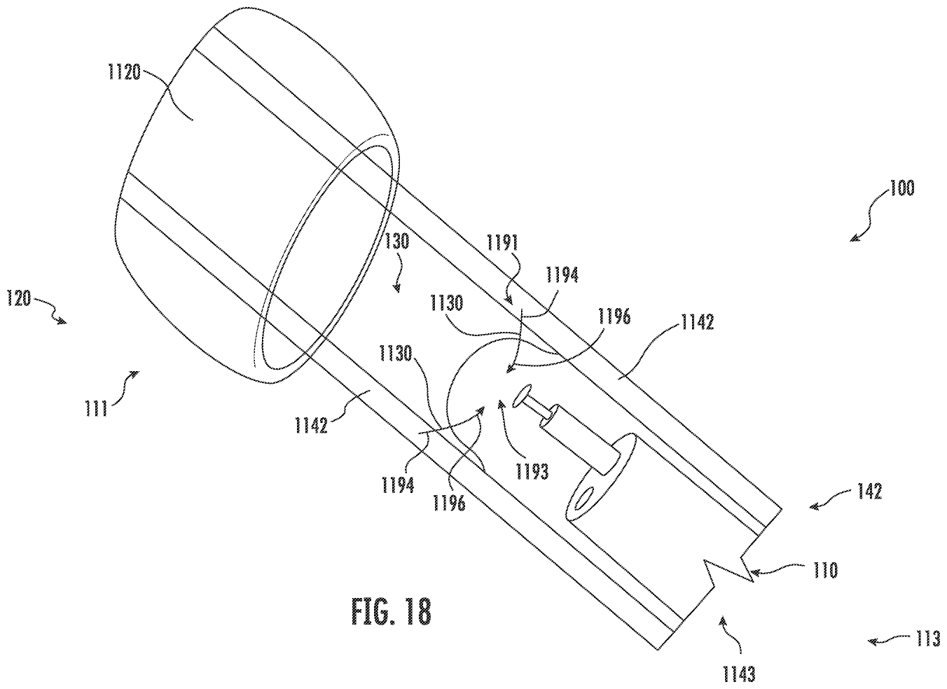

In the embodiment illustrated in FIG. 18, the lever 1130 includes one or more tethers 1194 (typically an elastic member or the like, such as generally described herein and as known in the art), with a first end 1191 coupled to or mounted on the stabilizer controller 1142 and a second end 1193 coupled to a target tissue fastener 1196. Thus, two or more target tissue fasteners 1196 may be coupled to different locations along the target tissue T to lift the tissue as desired. Each target tissue fastener 1196 is coupled to the stabilizer controller 1142 as well, typically on different portions of the stabilizer controller 1142 (e.g., different control tubes) on different sides of the target tissue T. The target tissue fastener 1196 may be repositionably or nonrepositionably coupled to the target tissue T for the lever 1130 to leverage the target tissue T. The target tissue fasteners 1196 may be manipulated, for instance, with graspers. Coupling of the lever 1130 to the stabilizer controller 1142 stabilizes the lever 1130 relative to the target tissue T to leverage the target tissue T as needed or desired. The stabilizer controller 1142 may also be manipulated to manipulate the lever 1130.

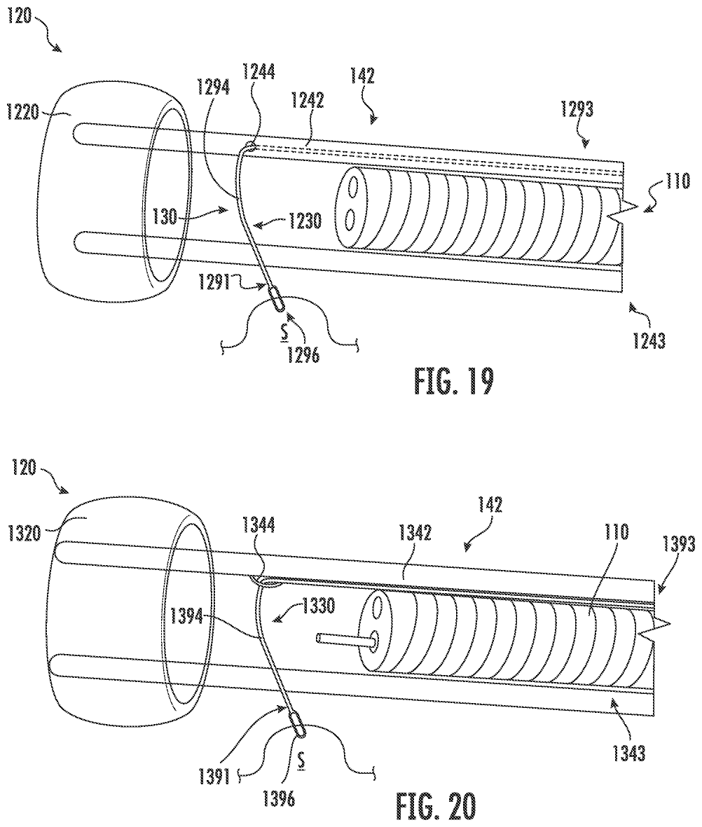

In the embodiment illustrated in FIG. 19, the lever 1230 is a tether 1294 (e.g., cable or cord or suture or the like, such as generally described herein and as known in the art) which passes through an opening 1244 in the stabilizer controller 1242 and runs through the stabilizer controller 1242 to a proximal end 113 of the delivery device 110. It will be appreciated that in this embodiment more than one stabilizer controller 1242 in the form of a tube is provided, with at least one being an inflation tube coupled to inflate the expandable stabilizer 1220 and one being a tube through which the tether 1294 runs. A distal end 1291 of the tether 1294 is coupled to a target tissue fastener 1296 repositionably or nonrepositionably coupled to the target tissue T, and the proximal end 1293 of the tether 1294 is accessible to the clinician (e.g., at the proximal end 113 of the delivery device 110) to manipulate the tether 1294 to leverage the lever 1230 and the target tissue T (coupled thereto) as desired or needed. It will be appreciated that the opening 1244 and the entry point/exit point of the tether 1294 into/out of the stabilizer controller 1242 may function as a direction-changing structure of the lever 130 by which the tether 1294 and target tissue fastener 1296 are leveraged when the proximal end 1293 of the tether 1294 is manipulated, thereby leveraging the target tissue T.

In the embodiment illustrated in FIG. 20, the lever 1330 includes a tether 1394 (e.g., cable or cord or suture or the like, such as generally described herein and as known in the art) which runs along the stabilizer controller 1342. A distal end 1391 of the tether 1394 is coupled to a target tissue fastener 1396 repositionably or nonrepositionably coupled to the target tissue T, and the proximal end 1393 of the tether 1294 is accessible to the clinician to manipulate the tether 1294 to leverage the lever 1230 and the target tissue T (coupled thereto) as desired or needed. A direction changing structure 1344 may be provided to leverage the tether 1394 when the proximal end 1393 of the tether 1394 is manipulated. In the embodiment illustrated in FIG. 20, the direction changing structure 1344 is a structure with an opening through which the tether 1394 passes, such as a loop or pulley or eyelet. In one embodiment, the direction changing structure 1344 is mounted on (e.g., to the exterior of) the stabilizer controller 1342.

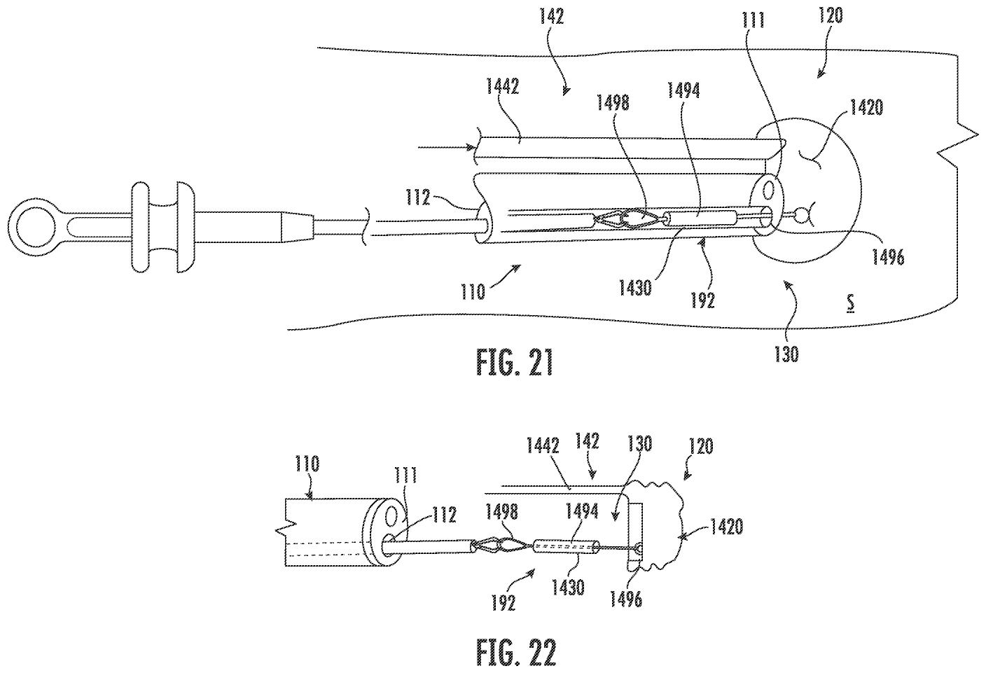

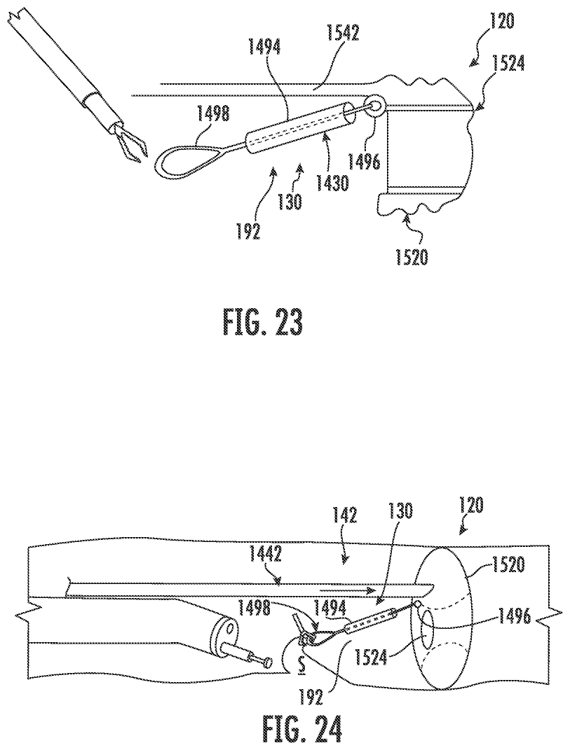

Instead of being mounted over the delivery device 110, as in the embodiments of FIGS. 16-20, the stabilizer 120 of a stabilization and leverage device 100 formed in accordance with general principles of the present disclosure to be delivered distal to the target tissue site S may be delivered to the target tissue site S by running in front of the delivery device 110, as in the embodiments of FIGS. 21-24. For instance, the stabilizer 120 may be delivered via or in the form or configuration of a releasable cap over the distal end 111 of the delivery device 110. For example, in the embodiment illustrated in FIGS. 21 and 22, the stabilizer 120 is in the form of an inflatable balloon stabilizer 1420. Preferably, the stabilizer 1420 is a clear balloon to allow visualization therethrough to facilitate navigation of the delivery device 110 to the target tissue site S. In some embodiments, the stabilizer 1420 has a delivery device interface 1444, such as a distally-facing seat formed in the distal side of the stabilizer 1420 shaped and configured to receive a distal end 111 of the delivery device 110 so that the stabilizer 1420 is seated on the distal end 111 of the delivery device 110 during navigation and delivery to the target tissue site S. In the embodiment of FIGS. 23 and 24, the stabilizer 120 is in the form of an inflatable balloon stabilizer 1520, similar to the inflatable balloon stabilizer 1420 of the embodiment of FIGS. 21 and 22, except toroidal in form with a passage or opening 1524 therethrough to permit navigation of an endoscope or other tool therethrough, if desired. A separately formed center ring (e.g., formed of a material more rigid than the material of the stabilizer 1420) may be provided in the opening 1524.

A stabilizer controller 142, such as in the form of an inflation tube 1442, 1542, runs along the delivery device 110 (either alongside and outside the delivery device 110 or along and within the working channel 112 thereof). Once the distal end 111 of the delivery device 110 is at the target tissue site S, the stabilizer 1420, 1520 is distally advanced then expanded (e.g., inflated by passing a fluidic, such as air or fluid, through the stabilizer controller 1442, 1542) to stabilize the target tissue site S and to allow independent operation of the delivery device 110 and any tools passed through the working channel 112 thereof.

In the embodiments of FIGS. 21-24, the lever 1430 includes a tissue traction device 192 delivered through the working channel 112 of the delivery device 110. In some embodiments, the tissue traction device 192 is preloaded on the stabilizer 120. It will be appreciated that in FIG. 21, the delivery device 110 is shown partially cut away to show a tissue traction device 192 carried by a stabilizer 1420. In other words, the stabilizer 1420 is preloaded with a tissue traction device 192 as the stabilizer 1420 is delivered via the delivery device 110 (e.g., loaded on the front of the delivery device 110 with the tissue traction device 192 carried within the working channel 112 of the delivery device 110). Once the stabilizer 120 has been deployed and stabilizing target tissue T, the tissue traction device 192 can be coupled with the target tissue T (e.g., as illustrated in FIG. 24) to leverage the target tissue T. For instance, in embodiments in which the tissue traction device 192 includes a tether 1494 (e.g., an elastic member such as a rubber band or the like) and tissue fasteners 1496, 1498 (e.g., in the form of loops), one tissue fastener 1496 may be coupled to (e.g., preloaded on) the stabilizer 1420, the other tissue fastener 1498 may be coupled to (e.g., through the use of a clip and/or grasper, such as illustrated in FIG. 23) the target tissue T, and the tether 1494 can apply traction to the target tissue T.

It will be appreciated that a stabilization and leverage device and system in accordance with principles of the present disclosure may be used for procedures within or adjacent to various body organs such as an esophagus, a heart, a stomach, a pelvic area, a bladder, an intestine; or a body passage or lumen or cavity or orifice such as any portion of a gastrointestinal, urinary, or pulmonary tract.

A delivery device used herewith may be any suitable size, cross-sectional shape or area, and/or configuration permitting introduction and passage of medical instruments to the distal end of the delivery device. It is generally beneficial for the delivery device to be steerable, and the delivery device may have different areas of different flexibility or stiffness to promote steerability. The delivery device may include one or more working channels extending substantially longitudinally (axially) between the proximal end and the distal end of the delivery device. The delivery devices and/or overtubes associated therewith may be made from any suitable biocompatible material known to one of ordinary skill in the art and having sufficient flexibility to traverse non-straight or tortuous anatomy. Such materials include, but are not limited to, rubber, silicon, synthetic plastic, stainless steel, metal-polymer composite; metal alloys of nickel, titanium, copper cobalt, vanadium, chromium, and iron; superelastic or shape memory material such as nitinol (nickel-titanium alloy); different layers of different materials and reinforcements. Such materials may be made of or coated with a polymeric or lubricious material to enable or facilitate passage of a deliver device therethrough. In some embodiments, the working channels may be made of or coated with a polymeric or lubricious material to facilitate passage of the introduced medical instrument(s) through the working channel(s).

The medical devices, instruments, tools, etc. of the present disclosure are not limited, and may include a variety of medical devices, instruments, tools, etc., for accessing body passageways, including, for example, duodenoscopes, catheters, ureteroscopes, bronchoscopes, colonoscopes, arthroscopes, cystoscopes, hysteroscopes, and the like. Various medical devices, instruments, tools, etc., may be used in conjunction with a stabilization and leverage device formed in accordance with principles of the present disclosure. For instance, such instruments or tools may be used to perform a procedure or operation which is either diagnostic or therapeutic or both, such as grasping, resecting, dissecting, retracting, cutting, and/or otherwise manipulating tissue. Such instruments or tools include graspers (e.g., a rotatable grasping clip, such as a RESOLUTION™ clip device sold by Boston Scientific Corporation, with a pair of jaws/arms, etc.), cutting tool (e.g., knife, electrocautery device, scissors), snares, etc.

A stent used in a stabilization and leverage device as disclosed herein may be a braided and/or twisted lattice of wire(s), a helical or semi-helical spiral, and/or a plurality of undulating, corrugated, or sinusoidal rings. A stent used in a stabilization and leverage device as disclosed herein may be made at least partially of a shape-memory material such as, for example, a cobalt-chromium-nickel alloy like Elgiloy; synthetic plastics, stainless steel; superelastic metallic alloys of nickel and titanium (e.g., nitinol); copper; cobalt, vanadium, chromium, iron, or the like; other metal alloys; powdered metals; ceramics; thermal plastic composites; ceramic composites; or polymers; and/or any combination thereof.

A target tissue fastener used with a stabilizer and leverage device formed in accordance with principles of the present disclosure may be formed of one or more of the following: a metal, a wire, a plastic, or a generally elastic material.

Components of a lever used with a stabilizer and leverage device formed in accordance with principles of the present disclosure may be formed of plastic, metal, elastomer, and/or the same material as the stabilizer or the delivery device. The lever may be coated by duraskin, a PTFE “Teflon” based material, to allow a tensioner component to slide more easily, such as to be moved proximally, to apply tension to the target tissue.

All apparatuses and methods discussed herein are examples of apparatuses and/or methods implemented in accordance with one or more principles of this disclosure. These examples are not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure.

In the foregoing description and the following claims, the following will be appreciated. The phrases “at least one”, “one or more”, and “and/or”, as used herein, are open-ended expressions that are both conjunctive and disjunctive in operation. The term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The foregoing discussion has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. One skilled in the art will appreciate that the disclosure may be used with many modifications of structure, arrangement, proportions, materials, components, and otherwise, used in the practice of the disclosure, which are particularly adapted to specific environments and operative requirements without departing from the principles of the present disclosure. For example, elements shown as integrally formed may be constructed of multiple parts or elements shown as multiple parts may be integrally formed, the operation of elements may be reversed or otherwise varied, the size or dimensions of the elements may be varied. The presently disclosed embodiments are therefore to be considered in all respects as illustrative and not restrictive, the scope of the claimed invention being indicated by the appended claims, and not limited to the foregoing description.

The following claims are hereby incorporated into this Detailed Description by this reference, with each claim standing on its own as a separate embodiment of the present disclosure. In the claims, the term “comprises/comprising” does not exclude the presence of other elements or steps. Furthermore, although individually listed, a plurality of means, elements or method steps may be implemented by, e.g., a single unit or processor.

Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second”, etc., do not preclude a plurality. Reference signs in the claims are provided merely as a clarifying example and shall not be construed as limiting the scope of the claims in any way.

What is claimed is:

1. A stabilization and leverage device comprising:
a stabilizer configured for mounting on a delivery device; and
a lever associated with said stabilizer and extending between a target tissue and said stabilizer to leverage the target tissue;
wherein said stabilizer contacts and stabilizes the target tissue independently of the distal end of the delivery device to allow independent free movement of the delivery device and instruments extended therethrough relative to said stabilizer and the target tissue.

2. The stabilization and leverage device of claim 1, wherein said lever comprises one or more of: a support elevator; a tensioner or controller; a lever tool; a direction-changing structure; a tissue traction device.

3. The stabilization and leverage device of claim 1, wherein:
a stabilizer passage extends from a proximal end to a distal end of said stabilizer; and
said lever includes a lever component extending from a first end coupled with the target tissue, through said stabilizer passage, to a second end controllable from a proximal end of the delivery device to leverage the target tissue.

4. The stabilization and leverage device of claim 3, wherein:
said lever component includes a lever tool extending through said stabilizer passage;
said lever includes a support elevator associated with a passage through said stabilizer;
said support elevator comprises one of: a flapper; a support and lever controller coupled to said support; a working channel; or a secondary inflatable balloon; and
said support elevator is configured to support and leverage said lever tool.

5. The stabilization and leverage device of claim 1, wherein:
said stabilizer is an expandable stabilizer;
said lever includes a tensioner extending between said expandable stabilizer and the target tissue; and
expansion of said expandable stabilizer leverages said lever and the target tissue.

6. The stabilization and leverage device of claim 1, wherein:
said stabilization and leverage device further includes a stabilizer controller associated with said stabilizer to facilitate manipulation of said stabilizer from a proximal end of the delivery device; and
said lever comprises at least one of: a tether having a first end coupled to the target tissue and a second end coupled to the stabilizer controller; a tether extending from a first end coupled to the target tissue, through the stabilizer controller, and to a second end accessible at a proximal end of the delivery device for manipulation of said tether to leverage the target tissue; or a tether extending from a first end coupled to the target tissue, through a direction-changing structure on said stabilizer controller, and along said stabilizer controller to a second end accessible at a proximal end of the delivery device for manipulation of said tether to leverage the target tissue.

7. The stabilization and leverage device of claim 1, wherein said stabilizer has a delivery device interface shaped and configured to receive a distal end of the delivery device during deployment of said stabilization and leverage device with the delivery device to a target tissue site.

8. The stabilization and leverage device of claim 7, wherein said lever comprises a lever component loaded through a working channel of the delivery device and preloaded on a proximal end of said stabilizer mounted on a distal end of the delivery device.

9. The stabilization and leverage device of claim 7, wherein said stabilizer has a ring opening therethrough shaped and configured to permit passage of an instrument therethrough.

10. A stabilization and leverage system for delivery to a target tissue site, said stabilization and leverage system comprising:
a delivery device;
a stabilizer configured for mounting on said delivery device; and
a lever associated with said stabilizer and extendable between a target tissue and said stabilizer to leverage the target tissue;
wherein said stabilizer stabilizes the target tissue independently of the distal end of said delivery device to allow independent movement of said delivery device and instruments extended therethrough relative to said stabilizer and the target tissue.

11. The stabilization and leverage system of claim 10, wherein:
said stabilizer includes a stabilizer passage associated therewith; and
said lever includes a lever component passing from a proximal end of said delivery device and through said stabilizer passage to be coupled to target tissue distal to said delivery device to leverage the target tissue.

12. The stabilization and leverage system of claim 10, wherein:
said lever includes a support elevator associated with said stabilizer passage;
said stabilizer passage is configured for passage of a tensioner therethrough for coupling with the target tissue; and
said support elevator and said stabilizer passage leverage the tensioner to leverage the target tissue.

13. The stabilization and leverage system of claim 12, wherein:
said lever component includes a lever tool extending through said stabilizer passage;
said lever includes a support elevator associated with a passage through said stabilizer;
said support elevator comprises one of: a flapper; a support and lever controller coupled to said support; a working channel; or a secondary inflatable balloon; and
said support elevator is configured to support and leverage said lever tool.

14. The stabilization and leverage system of claim 10, wherein:
said stabilizer is an expandable stabilizer with a stabilizer passage associated therewith;
said lever includes a tensioner extending between said expandable stabilizer and the target tissue; and
expansion of said expandable stabilizer leverages said lever and the target tissue.

15. The stabilization and leverage system of claim 10, wherein:
said stabilization and leverage device further includes a stabilizer controller associated with said stabilizer to facilitate manipulation of said stabilizer from a proximal end of the delivery device; and
said lever comprises at least one of: a tether having a first end coupled to the target tissue and a second end coupled to said stabilizer controller; a tether extending from a first end coupled to the target tissue, through said stabilizer controller, and to a second end accessible at a proximal end of the delivery device for manipulation of said tether to leverage the target tissue; or a tether extending from a first end coupled to the target tissue, through a direction-changing structure on said stabilizer controller, and along said stabilizer controller to a second end accessible at a proximal end of the delivery device for manipulation of said tether to leverage the target tissue.

16. The stabilization and leverage system of claim 10, wherein said stabilizer has a delivery device interface shaped and configured to receive a distal end of said delivery device during deployment of said stabilization and leverage device with said delivery device to a target tissue site.

17. The stabilization and leverage system of claim 16, wherein said lever comprises a lever component loaded through a working channel of said delivery device and preloaded on a proximal end of said stabilizer mounted on a distal end of said delivery device.

18. A method of stabilizing a target tissue site and leveraging target tissue at the target tissue site, said method comprising:
delivering, with a delivery device, a stabilization and leverage device loaded over the delivery device;
deploying the delivery device to stabilize the target tissue independently of the distal end of the delivery device to allow independent free movement of the delivery device and instruments extended therethrough relative to the stabilizer and the target tissue; and
extending a lever between the target tissue and the stabilizer and associating the lever with the stabilizer to leverage the target tissue.

19. The method of claim 18, further comprising:
deploying the stabilizer proximal to the target tissue site and proximal to the distal end of the delivery device; and
extending the lever proximally from the target tissue site to the stabilizer and proximal to the distal end of the delivery device.

20. The method of claim 18, further comprising:
controlling the stabilizer via stabilizer controllers extending from the stabilizer to a proximal end of the delivery device;
deploying the stabilizer distal to the target tissue site and distal to the distal end of the delivery device;
extending the lever proximally from the target tissue site to the stabilizer controllers; and
leveraging the lever with the stabilizer controllers to leverage the target tissue.
